(19) United States
(12) Patent Application Publication
(10) Pub. No.: US 2002/0031502 A1
(43) Pub. Date: Mar. 14, 2002

(54) Method for gene transfection using synergistic combinations of cationic lipids and cationic polymers

(76) Inventors: Atso Raasmaja, Kuopio (FI); Pasi Lampela, Kuopio (FI); Pekka T. Mannisto, Kuopio (FI)

Correspondence Address: BIRCH STEWART KOLASCH & BIRCH, PO BOX 747, FALLS CHURCH, VA, 22040-0747, US

(21) Appl. No.: 09/987,156

(22) Filed: Nov. 13, 2001

(30) Foreign Application Priority Data
Mar. 7, 2000 (FI) 20001584

(51) Int. Cl. A61K 48/00; C12N 15/88; A61K 9/127

(52) U.S. Cl. 424/93.21; 435/455; 435/320.1; 424/450; 435/458

(57) ABSTRACT

The present invention relates to a synergistic method of gene transfection, including gene therapy of human diseases. The invention provides use and preparation of specific combinations of cationic lipids and cationic polymers for the transfection of nucleic acids into host cells. This invention includes also the use of described combinations in specific systems for gene transfection, expression, repair, activation, inhibition and regulation.

### FIELD OF THE INVENTION

The present invention relates to a synergistic method of gene transfection, including gene therapy of human diseases. The invention provides the use and preparation of specific combinations of cationic lipids (e.g., Dosper) and cationic polymers (e.g., PEIs or polyethylemimines) for the transfection of DNA or RNA or synthetic nucleic acids (including plain nucleic acids, genomic or nongenomic DNA, nonviral expression plasmids and viral vectors) into host cells in vitro and in vivo. This invention includes also the use of described combinations in any systems for gene transfection, expression, repair, activation, inhibition and regulation, when a specific combination of cationic lipids and cationic polymers is used for the procedure. In addition, the present invention includes any delivery of other molecules or compounds into the cells, especially negatively charged, when any combinations of cationic liposomes and cationic polymers are used for the procedure, especially in therapeutic aims.

### BACKGROUND OF THE INVENTION

Gene therapy is a potential technique for the treatment of genetic and acquired diseases. The aim of this therapy is to correct a pathophysiological dysfunction by an introduction of therapeutic genes into a patient's target cells¹. Gene therapy may have a better specificity and selectivity, and a longer duration when compared to a traditional drug treatment. It could also be possible to treat by gene therapy rather the cause than the symptoms of the diseases.

Viral and non-viral gene deliveries have been two basic methods to transfer the therapeutic genes into the host cells. In general, nonviral vectors have been less efficient than viral vectors. In the former, the use of viral vectors (e.g., adenoviral or retroviral vectors) has been efficient, although they have caused immunogenic and cytotoxic side effects². In the non-viral gene therapy, modified plasmids could be used instead of viral vectors. However, these plasmids are often large and negatively charged which properties reduce their ability to enter the target cells. Indeed, the use of nonviral gene transfection has been limited by the low transfection efficiency. Therefore, different synthetic vectors, e.g., polylysine and its conjugates³, polyethylenimines (PEI)⁴, dendrimers⁵ and cationic lipids ^{1,6}, have been developed to increase the transfection efficiency.

Cationic lipids have been tested in the gene transfection. As an example, we have used Dosper Liposomal Transfection Reagent (1,3-di-oleoyloxy-2-(6-carboxy-spermyl)-propylamid, Boehiringer Mannheim, Germany) which is a polycationic liposomal compound. In general, the cationic liposomes form electrostatic complexes with negatively charged DNA, and these complexes can enter the cell via an endocytotic pathway⁷. The liposome-DNA complex destabilizes the endosomal membrane, and induces a flip-flop of anionic lipids from the cytoplasm-facing monolayer. These anionic lipids diffuse laterally into the liposome-DNA complex and form a charged neutral ion pair with the liposomes (cationic lipids). In this way, the liposome-DNA complex is broken and the DNA is released into the cytoplasm⁸.

PEIs are cationic polymers, and especially their branched forms are efficient vectors for delivering plasmids into cells⁴. Every third atom of each PEI molecule is an amino nitrogen that can be protonated. PEI has the ability to condense DNA⁹. In addition, PEI retains a substantial buffering capacity at virtually any pH¹⁰, protects DNA from endosomal degration⁴ and targets DNA into nucleus¹¹. When considering transfection efficiency, the ratio of PEI nitrogens to DNA phosphates (N/P ratio) is important, and maximal transfection efficiencies have been obtained with N/P ratios of 5-13.5¹². Transfection efficiency of PEI increases with the increase of molecular weight. The low molecular weight PEIs (600, 1200, 1800) have been virtually ineffective¹³. Large PEIs, like PEI 25K (average MW 25 000)^{10,11,14,15} and PEI 800K (average MW 800 000)^{4,10,14} have been successfully used in transfection studies. Large and small PEIs have also been used together to improve transfection efficiency

In general, cationic liposomes and polyethylenimines (PEI) have been successfully used in vitro in non-viral gene transfer. They have different mechanisms of action. Polycationic liposomes form complexes with DNA which then enter the cell via an endocytotic pathway. PEIs condense DNA and protect them from endosomal degradation thereby improving transfection efficiency. In earlier reports, only large PEI molecules (MW>20 000) have been effective in the gene transfection.

The transfection method described here uses the advantage of the synergism between cationic lipids and cationic polymers to potentiate significantly the transfection efficiency. This synergism may result from the different mechanisms of described compounds, therefore allowing a higher transfection efficiency of gene expression plasmids. The synergistic effect of PEI and cationic liposomes was a novel unexpected finding in the gene transfection.

### SUMMARY OF THE INVENTION

We have identified and characterized a new method of gene transfection which is based on the use of a combination of cationic lipids (e.g., Dosper liposomes) and cationic polymers (e.g. polyethylenimines). In this study, we have shown that when a PEI and a polycationic liposome, Dosper Liposomal Transfection Reagent, are combined, the transfection efficiency of a reporter gene lacZ can be greatly enhanced as seen as an increased β-galactosidase activity and X-gal staining in monkey fibroblastoma cells. This synergy was seen with all three PEIs studied (average MW 700, 2000 and 25 000) and even at low Dosper/DNA ratios.

A general object of the present invention is to provide a method to be used in in vitro and in vivo gene transfection.

A specific object of the present invention is the use of a combination of cationic lipids and cationic polymers in the gene transfection, especially Dosper and polyethylenimines.

A further object of the invention is a composition for transfecting a cell, which comprises one or more nucleic acid molecules, polycations or cationic polymers, and cationic liposomes or lipids.

Another specific object of the present invention is the use of small polyethylenimines (e.g. PEI 700 and PEI 2000) in the combination of cationic lipids and cationic polymers for the gene transfection.

Another specific object is the use of such amounts of reagents which are alone ineffective in the gene transfection. These amounts are varying from low and ineffective to high and effective. Especially, the synergism with Dosper and PEIs at concentration where they alone are ineffective.

Another specific object is the expression of a therapeutic gene in the cells of a subject. The expression is achieved by transfecting the cells with the help of the present method.

Another specific object of the present invention is the transfection and expression of any plasmids or vectors containing a therapeutic gene, to be used in the gene therapy of human diseases.

The polyethylenimines are used at equivalencies from 1 to 150 N/P, and other polycations are used at equivalent amounts and principle.

In general, the transfection reagents of the invention, i.e. polycations or cationic polymers and cationic liposomes or lipids are used in very low amounts, which are inefficient alone.

### DETAILED DESCRIPTION OF THE INVENTION

The invention will be described in more detail in the following experimental section of this specification, referring to the enclosed drawings, wherein

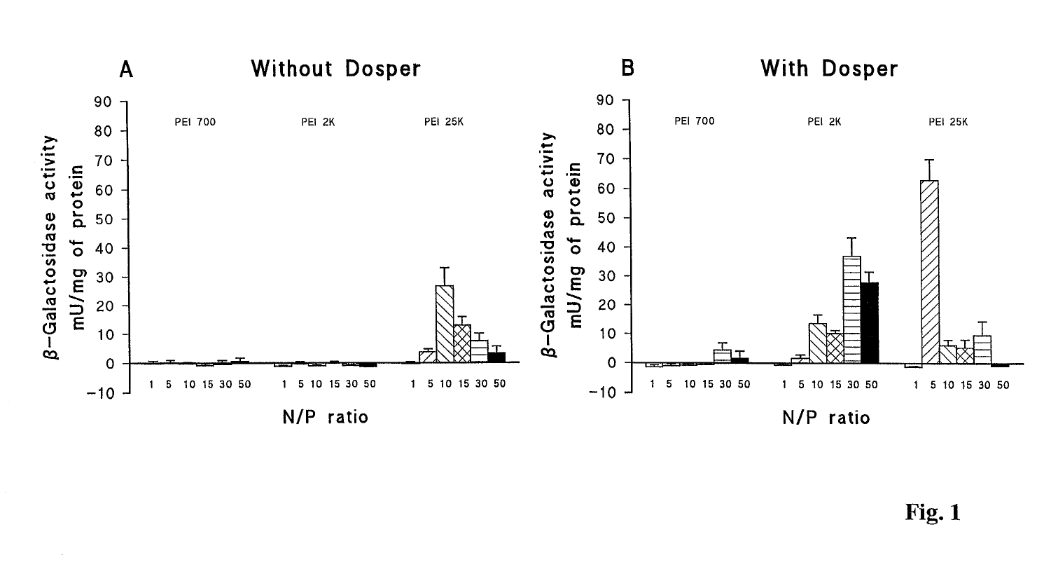

FIG. 1 shows the effect of addition of Dosper to the PEI-DNA complexes on the β-galactosidase activity. The cells were transfected with TkBPVlacZ plasmid (1 μg) complexed with three different PEIs (average MW 700, 2000 and 25 000) with different N/P ratios (1-50). The transfections were carried out without (A) or with (B) addition of Dosper (Dosper/DNA ratio of 1) to the PEI-DNA complexes. The cells were incubated in the transfection solution for 6 h and then in the growth medium for 42 h. The β-galactosidase activity was measured by ONPG assay. The values are means of β-galactosidase activity/mg protein ±SE, n=6. When only Dosper was used, β-galactosidase activity was 0.36+0.53 mU/mg protein.

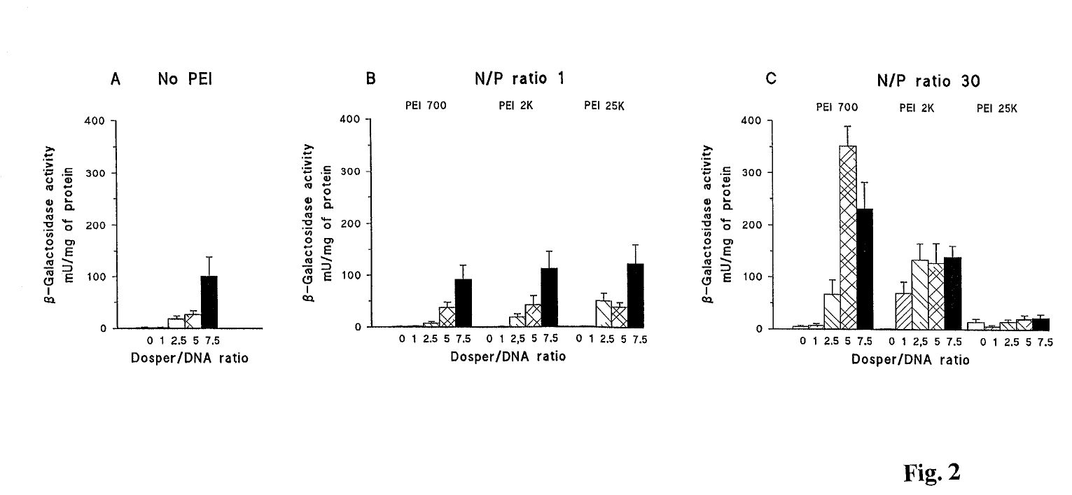

FIG. 2 shows the effect of different Dosper/DNA ratios on the β-galactosidase activity. The cells were transfected with 1 μg TkBPVlacZ plasmid at a Dosper/DNA ratios 0-7.5 (A) or DNA was condensed with PEIs at N/P ratios 1 (B) and 30 (C) before the addition of Dosper. The cells were incubated in the transfection solution for 6 hours and then in the growth medium for 42 h. The β-galactosidase activity was measured by ONPG assay. The values are the means of β-galactosidase activity/mg protein ±SE, n=6.

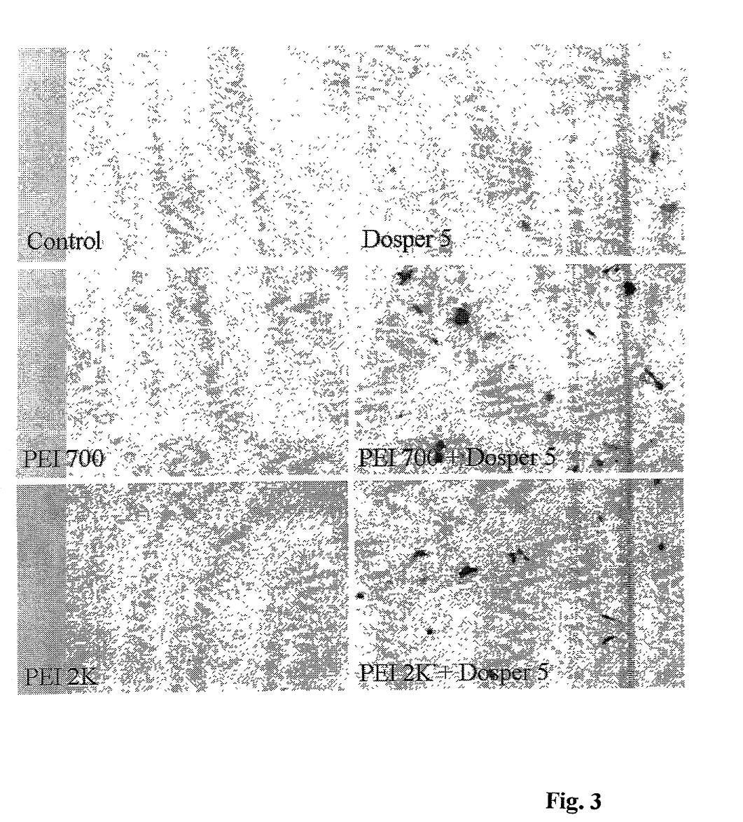

FIG. 3 shows the analysis of transfection efficiency by X-gal staining. The cells were transfected with 1 μg TkBPVlacZ plasmid complexed with PEI700 and PEI2K at the N/P ratio of 30 and/or Dosper at a Dosper/DNA ratios of 0, 2.5 and 5. The cells were incubated in the transfection solution for 6 h and then incubated in the growth medium for 42 h. After the incubation, the cells were washed with PBS and fixed with 4% paraformaldehyde for 15 min. Then, the cells were stained with X-gal (1 mg/ml) for 3 h at 37° C. and washed with PBS.

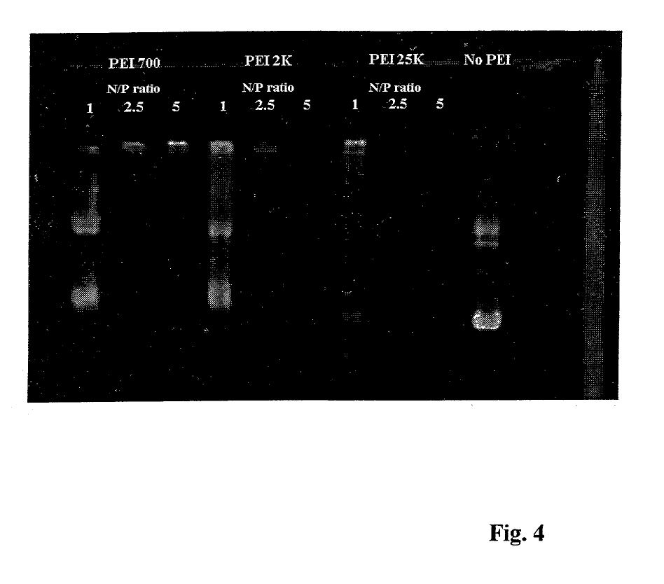

FIG. 4 shows the analysis of complex formation by agarose gel electrophoresis. TkBPVlacZ plasmids were complexed with PEIs (MW 700, 2000 and 25 000) at the N/P ratios 1, 2.5 and 5. With N/P ratios above 1, all the DNA was complexed and remained in the well. TkBPVlacZ plasmid (2 μg) was diluted with 150 mM NaCl to total volume of 30 μl. Different amounts of PEI were also diluted with NaCl to the total volume of 30 μl. After a 10 min incubation, the solutions were mixed and the PEI-DNA complexes were allowed to form during another 10 min incubation. Then, 14 μl of loading buffer was added and 17,5 μl (0,5 μg DNA) of each solution was loaded in a well for electrophoretic separation (0,6% agarose in 1x Tris-Acetate-EDTA buffer; electrophoresis at 28 V for 6 h). DNA was visualized with ethidium bromide.

### EXPERIMENTAL SECTION

Chemicals

Dulbecco's Modified Eagle Medium (DMEM), Foetal Bovine Serum and Penicillin-Streptomycin were purchased from Gibco BRL (U.K.), Dosper Liposomal Transfection Reagent and X-gal (5-bromo-4-chloro-3-indolyl-β-D-galactoside) from Boehringer Mannheim (Germany) and polyethylenimines and ONPG (o-nitrophenol-β-D-galactopyranoside) from Sigma-Aldrich (USA). The pTKBPVlacZ plasmid was synthesized by prof. Mart Ustav at University of Tartu, Estonia. The pTkBPVlacZ plasmid was produced in E. coli (DH5α) and purified using the commercial kits (Qiagen, Germany). All other chemicals were of cell culture and molecular biological quantity.

Gene Transfection

The pTKBPVlacZ expression plasmids (1 μg/well) were transfected in the subconfluent CV1-P cell cultures (a monkey fibroblastoma cell line). The cells were cultured in 24-well plates in the atmosphere containing 5% CO₂ at 37° C. The bacterial lacZ gene encoding the β-galactosidase enzyme was used as a reporter gene. Preparation of transfection mixtures were made separately for PEI, Dosper and PEI/Dosper combinations. First, 10 μg of pTKBPVlacZ plasmid was diluted to a final volume of 150 μl of 150 mM NaCl. For the PEI transfection mixture, 10 mM PEI was diluted to a final volume of 150 mM NaCl, incubated for 10 min, added to plasmid DNA dilution and incubated for another 10 min before the gene transfection. For the Dosper transfection mixture, Dosper was diluted at the ratio of 115 in 150 mM NaCl and incubated for 15 min before the use for gene transfection. In the case of PEI/Dosper combination, Dosper solution was added to the DNA-PEI mixture which was then incubated for an additional 15 min. For gene transfections, the transfection mixtures were pipetted dropwise to cell cultures with 1 ml of freshly added DMEM without serum and antibiotics. After 6 h exposure, the transfection mixture was replaced with 1 ml of fresh DMEM (with 9% Foetal Bovine Serum and 90 U Penicillin-Streptomycin). The cells were incubated further up to 42 h before analyses of the β-galactosidase activity.

ONPG Analysis

At the end of experiments, the cells were washed with PBS, lysed with 150 μl lysis reagent (25 mM glycylglycine, 15 mM MgSO₄, 4 mM EGTA, 1% Triton X-100, 1 mM DTT, 1 mM PMSF), and centrifuged at 13 000 rpm for 5 min (Eppendorf Centrifuge 5415C, Eppendorf-Netheler-Hinz, Germany). The activity of β-galactosidase was measured with ONPG assay from the supernatant: 20 μl of the supernatant, 80 μl H₂O and 100 μl of 2x β-gal solution (2 mM MgCl₂, 1 mM β-mercaptoethanol, 1.33 mg/ml ONPG in sodium phosphate buffer (0.2M)) were put in a 96-well plate and incubated up to 1 h at RT. In the ONPG assay, the detection is based on the cleavage of β-bond from ONPG by the β-galactosidase enzyme resulting in the yellow o-nitrophenol molecule. The reaction was stopped with 1M Na₂CO₃, when o-nitrophenol anionizes and absorbs lightwaves at 405 nm wavelength. Samples were analysed by measuring absorbance at 405 nM wavelength with the Bio-Tek Elx-800 microplate reader (Bio-Tek Instruments, USA) and KC-3 PC-program.

Protein Assay

Protein concentrations were measured with Bio-Rad Protein Assay (Coomassie Brilliant Blue, Bio-Rad Laboratories, USA). 15 μl of supernatant was diluted to 800 μl of H₂O, and 200 μl of Protein Assay Dye Reagent Concentrate was added. Absorbance was read at 595 nm wavelength using Hitachi U-2000 spectrophotometer.

Histochemistry

The X-gal staining was used for histochemical analysis of β-galactosidase enzyme. Therefore, the cells were washed with PBS, fixed with 4% paraformaldehyde (15 min, RT) and washed again twice with PBS. Then, the cells were incubated in the X-gal staining solution (X-gal 1 mg/ml, MgCl₂ 2, 5 mM K₃Fe(CN)₆, 5 mM K₄Fe(CN₆)x3H₂O, 0.01% sodiumdeoxycholate, 0.02% Nonidet P-40) for 3 h at +37° C. The activity of β-galactosidase was detected as a blue color of 3,5′-dichromo-4,4′-dichloroindigo molecule resulting from the cleavage of X-gal substrate by the β-galactosidase in the transfected cells. After X-gal staining, the cells were washed with PBS and photographed with Nikon Diaphot 300 microscope and Nikon F-601 camera.

Gel Electrophoresis

The PEI-DNA complexes were prepared as described earlier. For electrophoresis, 0.5 μg DNA was pipetted into an agarose gel (0.6% agarose in 1x Tris-Acetate-EDTA buffer; electrophoresis at 28 V for 6 h). The gels were stained in ethidium bromide solution (0.5 mg EtBr in 1 l H₂O) for 30 min at RT to visualize DNA after electrophoresis.

Results

The role of PEI/DNA ratios on the transfection efficiency of the PEI/Dosper combination

In the first experiments, the effect of different N/P ratios of PEI/DNA complexes was studied on the transfection efficiency of the PEI/Dosper combination. Here, the transfection efficiency was measured as a total β-galactosidase activity in the cell extracts using the calorimetric ONPG assay.

Initially, we studied the effect of Dosper alone, three PEIs (700, 2K, 25K) alone and their different combinations on the transfection efficiency in the CV1-P cell cultures (FIG. 1). Therefore, each PEI was studied at varying N/P ratios with and without Dosper using a constant Dosper/DNA ratio, to find out if there would be any combinatory potentiation of transfection efficiency. We used Dosper alone at the Dosper/DNA ratio of 1, each of three PEIs alone at six N/P ratios varying from 1-50 or different combinations of Dosper and PEIs.

In these experiments, Dosper, PEI 700 and PEI 2K were all ineffective when they were used alone for gene transfection, whereas PEI 25K had a significant transfection efficiency. However, the transfection efficiency of PEI 700 was slightly potentiated with Dosper at the N/P ratios of 30and 50. Furthermore, when PEI 2K was used together with Dosper, the transfection efficiency was significantly improved especially at the N/P ratio of 30, while the smaller N/P ratios of 1 and 5 produced no or only a weak potentiation of β-galactosidase activity. This effect was even higher than that achieved by PEI 25K which itself produced a significant rise in β-galactosidase activity especially at the N/P ratio of 10. When Dosper was added to PE125K/DNA complex, the transfection efficiency was still enhanced, and the highest effect was seen at the N/P ratio of 5.

The role of Dosper/DNA ratios on the transfection efficiency of the PEI/Dosper combination

In the second experiment, the effect of different Dosper/DNA ratios was studied on the transfection efficiency of the PEI/Dosper combination (FIG. 2). Therefore, the N/P ratios of 1 and 30 were selected for each PEI, and different amounts of Dosper (Dosper/DNA ratios of 0-7.5) were added to PEI/DNA complexes.

When the cells were transfected using Dosper alone (i.e. without PEIs), the best β-galactosidase activity was achieved at the Dosper/DNA ratio of 7.5. When the combination of PEI/Dosper was used at the N/P ratio of 1 for PEI/DNA complexes, no significant changes were seen in the transfection efficiency with any of PEIs (700, 2K and 25K) in comparison to Dosper alone. However, for PEI 700 at the N/P ratio of 30, the transfection efficiency was significantly higher with the combination of PEI/Dosper. The highest potentiation was obtained at the Dosper/DNA ratio of 5, and was more than 3-fold compared to the best results achieved with Dosper alone. On the other hand, for PEI 2K at the NIP ratio of 30, the transfection efficiency was not altered with the combination of PEI/Dosper when compared to the plain Dosper. However, now the maximum β-galactosidase activity was achieved with somewhat lower Dosper/DNA ratios (2.5 and 5) than with Dosper alone (7.5). The use of PEI 25K at the N/P ratio of 30 did not enhance the transfection efficiency of PEI/Dosper combination compared to Dosper or PEI 25K alone.

The Effect of PEI/Dosper Combination on the Number of Transfected Cells

The number of transfected cells was measured using the histochemical X-gal staining. When Dosper was added to PEIIDNA complexes, the number of blue-colored cells (indicating successful lacZ gene transfections and expressions) was increased in comparison to PEI or Dosper alone (FIG. 3). There was no staining seen in any cells which were transfected only with PEI 700 or PEI 2K at the N/P ratio 30. In the Dosper-mediated gene transfection at the Dosper/DNA ratio of 5, there was less than 1% of the cells were stained. The number of stained cells increased significantly, when PEI 700 or 2K at the N/P ratio of 30, was used to condense the plasmid DNA prior addition of Dosper, showing the averages of with 4.8±0.8% for PEI 700 and 4.3±1% for PEI 2K (data not shown).

Analysis on the Formation of the PEI/DNA Complexes at Different N/P Ratios

Agarose gel electrophoresis was used to study the ability of PEIs to condense DNA, and the relationship of complex formation to the potentiated transfection efficiency bof the PEI/Dosper combinations. Each PEI at the N/P ratios >2.5 could complex all DNA since all the DNA was staying in the wells, whereas at the N/P ratio of 1, DNA was not totally complexed which was seen as the gel electrophoretic movement of free DNA (FIG. 4).

### REFERENCES

1 Murray K D et al. Cationic liposome-mediated DNA transfection in organotypic explant cultures of the ventral mesencephalon. Gene Ther 1999; 6: 190-197.

2 Mulligan R C. The basic science of gene therapy. Science 1993; 260: 926-932.

3 Wagner E, Cotten M, Foisner R, Bimstiel M L. Transferrin-polycation-DNA complexes: the effect of polycations on the structure of the complex and DNA delivery to cells. Proc Natl Acad Sci USA 1991; 88: 4255-4259.

4 Boussif O et al. A versatile vector for gene and oligonucleotide transfer into cells in culture and in vivo: polyethylenimine. Proc Natl Acad Sci U S A 1995; 92: 7297-7301.

5 Kukowska-Latallo J F et al. Efficient transfer of genetic material into mammalian cells using Starburst polyamidoamine dendrimers. Proc Natl Acad Sci U S A 1996; 93: 4897-4902.

6 Zabner J et al. Cellular and molecular barriers to gene transfer by a cationic lipid. J Biol Chem 1995; 270: 18997-19007.

7 Legendre J Y, Szoka F C Jr. Delivery of plasmid DNA into mammalian cell lines using pH-sensitive liposomes: comparison with cationic liposomes. Pharm Res 1992; 9: 1235-1242.

8 Xu Y, Szoka F C Jr. Mechanism of DNA release from cationic liposome/DNA complexes used in cell transfection. Biochemistry 1996; 35: 5616-5623.

9 Dunlap D D, Maggi A, Soria M R, Monaco L. Nanoscopic structure of DNA condensed for gene delivery. Nucleic Acids Res 1997; 25: 3095-3101.

10 Abdallah B et al. A powerfiil nonviral vector for in vivo gene transfer into the adult mammalian brain: polyethylenimine. Hum Gene Ther 1996; 7: 1947-1954.

11 Pollard H et al. Polyethylenimine but not cationic lipids promotes transgene delivery to the nucleus in mammalian cells. J Biol Chem 1998; 273: 7507-7511.

12 Godbey W T, W u K K, Mikos A G. Poly(ethylenimine) and its role in gene delivery. J Controlled Release 1999; 60:149-160.

13 Godbey W T, W u K K, Mikos A G. Size matters: Molecular weight affects the efficiency of poly(ethylenimine) as a gene delivery vehicle. J Biomed Mater Res 1999; 45: 268-275.

14 Boletta A et al. Nonviral gene delivery to the rat kidney with polyethylenimine. Hum Gene Ther 1997; 8: 1243-1251.

15 Goula D et al. Polyethylenimine-based intravenous delivery of transgenes to mouse lung. Gene Ther 1998; 5: 1291-1295.

16 Godbey W T, W u K K, Hirasaki G J, Mikos A G. Improved packing of poly(ethylenimine)/DNA complexes increases transfection efficiency Gene Ther 1999; 6: 1380-1388.

17 Zanta M A, Boussif O, Adib A, Behr J P. In vitro gene delivery to hepatocytes with galactosylated polyethylenimine. Bioconjug Chem 1997; 8: 839-844.

1. A composition for transfecting a cell, which comprises one or more nucleic acid molecules, polycations or cationic polymers, and cationic liposomes or lipids.

2. The composition of claim 1, wherein said composition comprises two or more polycations or cationic polymers and cationic liposomes or lipids as transfection reagents.

3. The composition of claim 1, wherein said cationic polymers comprise polyethylenimines.

4. The composition of claim 1, wherein said cationic liposomes comprise a Dosper Liposomal Transfection Reagent.

5. The composition of claim 3, wherein said polyethylenimines are selected from polyethylenimines 700, 2K and 25K.

6. The composition of claim 3, wherein said polyethylenimines are especially low molecular weight polyethylenimines being ineffective alone and effective in combinations.

7. The composition of any one of claims 2 to 6, wherein the amount of transfection reagents is very low being inefficient when used alone.

8. The composition of claim 1, wherein the nucleic acids comprise plasmid DNA molecules containing one or more genes.

9. The composition of claim 4, wherein Dosper Liposomal Transfection Reagent is used at the charge ratios of liposome/DNA from 0.5 to 50.

10. The composition of claim 1, wherein the nucleic acids comprise DNA, RNA and synthetic nucleic acids including plain nucleic acids, genomic DNA, nongenomic DNA, nonviral expression plasmids, viral vectors and oligonucleotides.

11. The composition of any one of the claims 1 to 10 for use in transfecting a host cell of a subject with the nucleic acids comprising DNA, RNA and synthetic nucleic acids including plain nucleic acids, genomic DNA, nongenomic DNA, nonviral expression plasmids, viral vectors and oligonucleotides.

12. The composition of any one of the claims 1 to 10 for use in transfecting a host cell of a subject with a DNA plasmid containing one or more specific genes.

13. Use of the composition of any one of the claims 1 to 12 for synergistic potentiation of transfection efficiency.
